(19) United States
(12) Patent Application Publication
(10) Pub. No.: US 2024/0124567 A1
(43) Pub. Date: Apr. 18, 2024

(54) Thermoresponsive or Shear-Thinning Injectable Gel Compositions for Treatment of Arthritis

(71) Applicant: GLOBUS MEDICAL, INC., AUDUBON, PA (US)

(72) Inventors: Bipin Gaihre, King of Prussia, PA (US); Allison Adams, Conshohocken, PA (US); Archana Bhat, Phoenixville, PA (US); Jason Leigl, Collegeville, PA (US)

(21) Appl. No.: 18/045,981

(22) Filed: Oct. 12, 2022

(51) Int. Cl. C07K 16/24; A61K 9/00; A61K 9/14; C07K 16/22

(52) U.S. Cl. CPC C07K 16/24; A61K 9/0019; A61K 9/14; C07K 16/22

(57) ABSTRACT

The present invention relates to a thermoresponsive or shear-thinning gel composition comprising a recombinant factor (i.e., interleukin-1 receptor antagonist protein (IRAP) and/or platelet derived growth factor (PDGF)) and a carrier solution comprising at least one selected from hyaluronic acid (HA), chondroitin sulfate (CS), methyl cellulose (MC), and/or two-dimensional silicate nanomaterial. In some embodiments, the composition is an anti-inflammatory composition. Thus, in some embodiments, the present invention also provides a method of treating an inflammatory disease or disorder, such as osteoarthritis, using said composition.

### BACKGROUND OF THE INVENTION

Osteoarthritis (OA), the most common form of arthritis, is a leading cause of disability especially among older adults. Corticosteroids and hyaluronic acids (HA) are two available non-tissue derived solutions to alleviate the pain due to OA. Autogenic platelet rich plasma (PRP) and bone marrow aspirate concentrate (BMAC) have shown efficacy in pain reduction to a larger extent. However, both PRP and BMAC require invasive procedures to harvest blood and bone marrow, increasing patient discomfort and likelihoods of infection. Additionally, the harvested tissue needs further processing to extract the PRP and BMAC inside the surgical room, making the procedure unnecessarily intricate and space-limiting.

Thus, there is a need in the art for easy to use, injectable compositions for localized treatment of various inflammatory diseases or disorders, such as osteoarthritis. The present invention satisfies this unmet need.

### SUMMARY OF THE INVENTION

A thermoresponsive composition is described. The composition includes a recombinant factor such as interleukin-1 receptor antagonist protein (IRAP) and platelet derived growth factor (PDGF), and a carrier solution, where the carrier solution includes a solvent and at least one of hyaluronic acid (HA), chondroitin sulfate (CS), methyl cellulose (MC), and a two-dimensional silicate nanomaterial. In some embodiments, the recombinant factor includes IRAP and PDGF. In some embodiments, the composition includes an inorganic component. In some embodiments, the composition does not include an inorganic component. In some embodiments, the composition includes a carrier solution comprising a two-dimensional silicate nanomaterial and at least one selected from the group consisting of HA, CS, and MC. In some embodiments, the two-dimensional silicate nanomaterial is Laponite® (LAP). In some embodiments, the composition includes a carrier solution comprising between about 0.05% to about 2% w/v of LAP and between about 0.2% to about 1.5% w/v of HA. In some embodiments, the composition comprises a carrier solution comprising between about 1% to about 5% w/v of CS and between about 5% to about 20% w/v of MC. In some embodiments, the composition gels at physiological temperatures. In some embodiments, the composition is a liquid at between 0° C. and room temperature. In some embodiments, the composition is an injectable liquid composition. In some embodiments, the composition is an anti-inflammatory composition. In some embodiments, the composition prevents or reduces HA degradation, prevents or reduces ECM degradation, promotes chondrocyte growth, promotes proliferation to accelerate new ECM synthesis, or any combination thereof.

Also described are methods of treating osteoarthritis, preventing or reducing HA degradation, preventing or reducing ECM degradation, promoting chondrocyte growth, promoting proliferation to accelerate new ECM synthesis, or any combination thereof in a subject in need thereof. The method includes the step of administering to the subject a therapeutic effective amount of any of the compositions described herein. In some embodiments, the composition is administered to the subject via an injection. In some embodiments, the composition prevents or reduces HA degradation, prevents or reduces ECM degradation, promotes chondrocyte growth, promotes proliferation to accelerate new ECM synthesis, or any combination thereof at the injection site. In some embodiments, the composition is formulated less than 1 hour prior to the administration to the subject.

### BRIEF DESCRIPTION OF THE DRAWINGS

The following detailed description of various embodiments of the invention will be better understood when read in conjunction with the appended drawings. For the purpose of illustrating the invention, there are shown in the drawings illustrative embodiments. It should be understood, however, that the invention is not limited to the precise arrangements and instrumentalities of the embodiments shown in the drawings.

FIG. 1 is a schematic representation of an exemplary therapeutic formulation for osteoarthritis using a first carrier option.

FIG. 2 is a flowchart of the therapeutic formulation for osteoarthritis using the first carrier option.

FIG. 3 is a schematic representation of an exemplary therapeutic formulation for osteoarthritis using a second carrier option.

### DETAILED DESCRIPTION

### Definitions

As used herein, each of the following terms has the meaning associated with it in this section. Unless defined elsewhere, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described.

The articles “a” and “an” are used herein to refer to one or to more than one (i.e., to at least one) of the grammatical object of the article. By way of example, “an element” means one element or more than one element.

The term “about” will be understood by persons of ordinary skill in the art and will vary to some extent depending on the context in which it is used. As used herein when referring to a measurable value such as an amount, a temporal duration, and the like, the term “about” is meant to encompass variations of ±20% or ±10%, more preferably ±5%, even more preferably ±1%, and still more preferably ±0.1% from the specified value, as such variations are appropriate to perform the disclosed methods.

The terms “patient,” “subject,” “individual,” and the like are used interchangeably herein, and refer to any animal, or cells thereof whether in vitro or in situ, amenable to the methods described herein. In certain non-limiting embodiments, the patient, subject, or individual is a mammal, non-human mammal, primate, mouse, rat, pig, horse, ferret, dog, cat, cattle, or human.

A “disease” is a state of health of an animal wherein the animal cannot maintain homeostasis, and wherein if the disease is not ameliorated then the animal's health continues to deteriorate. In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

A disease or disorder is “alleviated” if the severity of a sign or symptom of the disease or disorder, the frequency with which such a sign or symptom is experienced by a patient, or both, is reduced.

The terms “treatment”, “treating” and the like are used herein to generally mean obtaining a desired pharmacological and/or physiological effect. The effect may be prophylactic in terms of completely or partially preventing a disease or symptom thereof and/or may be therapeutic in terms of partially or completely curing a disease and/or adverse effect attributed to the disease. The term “treatment” as used herein covers any treatment of a disease in a subject and includes: (a) preventing a disease related to an undesired immune response from occurring in a subject which may be predisposed to the disease; (b) inhibiting the disease, i.e., arresting its development: or (c) relieving the disease, i.e., causing regression of the disease.

The term “derivative” refers to a small molecule that differs in structure from the reference molecule but may retain or enhance the essential properties of the reference molecule and may have additional properties. A derivative may change its interaction with certain other molecules relative to the reference molecule. A derivative molecule may also include a salt, an adduct, tautomer, isomer, or other variant of the reference molecule.

The term “pharmaceutically acceptable salt” refers to any pharmaceutically acceptable salt, which upon administration to the patient is capable of providing (directly or indirectly) a compound as described herein. Such salts preferably are acid addition salts with physiologically acceptable organic or inorganic acids. Examples of the acid addition salts include mineral acid addition salts such as, for example, hydrochloride, hydrobromide, hydroiodide, sulphate, nitrate, phosphate, and organic acid addition salts such as, for example, acetate, trifluoroacetate, maleate, fumarate, citrate, oxalate, succinate, tartrate, malate, mandelate, methane sulphonate and p-toluenesulphonate. Examples of the alkali addition salts include inorganic salts such as, for example, sodium, potassium, calcium and ammonium salts, and organic alkali salts such as, for example, ethylenediamine, ethanolamine, N,N-dialkylenethanolamine, triethanolamine and basic amino acids salts. However, it will be appreciated that non-pharmaceutically acceptable salts also fall within the scope of the invention since those may be useful in the preparation of pharmaceutically acceptable salts. Procedures for salt formation are conventional in the art.

The terms “effective amount” and “pharmaceutically effective amount” refer to a sufficient amount of an agent to provide the desired biological result. That result can be reduction and/or alleviation of a sign, symptom, or cause of a disease or disorder, or any other desired alteration of a biological system. An appropriate effective amount in any individual case may be determined by one of ordinary skill in the art using routine experimentation.

A “therapeutically effective amount” refers to that amount which provides a therapeutic effect for a given condition and administration regimen. In particular, “therapeutically effective amount” means an amount that is effective to prevent, alleviate or ameliorate symptoms of the disease or prolong the survival of the subject being treated, which may be a human or non-human animal. Determination of a therapeutically effective amount is within the skill of the person skilled in the art.

As used herein, the term “pharmaceutical composition” refers to a mixture of at least one compound of the invention with other chemical components and entities, such as carriers, stabilizers, diluents, dispersing agents, suspending agents, thickening agents, and/or excipients. The pharmaceutical composition facilitates administration of the compound to an organism.

“Pharmaceutically acceptable” refers to those properties and/or substances which are acceptable to the patient from a pharmacological/toxicological point of view and to the manufacturing pharmaceutical chemist from a physical/chemical point of view regarding composition, formulation, stability, patient acceptance and bioavailability. “Pharmaceutically acceptable carrier” refers to a medium that does not interfere with the effectiveness of the biological activity of the active ingredient(s) and is not toxic to the host to which it is administered.

As used herein, the term “pharmaceutically acceptable carrier” means a pharmaceutically acceptable material, composition or carrier, such as a liquid or solid filler, stabilizer, dispersing agent, suspending agent, diluent, excipient, thickening agent, solvent or encapsulating material, involved in carrying or transporting a compound useful within the invention within or to the patient such that it may perform its intended function. Typically, such constructs are carried or transported from one organ, or portion of the body, to another organ, or portion of the body. Each carrier must be “acceptable” in the sense of being compatible with the other ingredients of the formulation, including the compound useful within the invention, and not injurious to the patient. Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; surface active agents; alginic acid; pyrogen-free water; isotonic saline; Ringer's solution; ethyl alcohol; phosphate buffer solutions; and other non-toxic compatible substances employed in pharmaceutical formulations. As used herein, “pharmaceutically acceptable carrier” also includes any and all coatings, antibacterial and antifungal agents, and absorption delaying agents, and the like that are compatible with the activity of the compound useful within the invention and are physiologically acceptable to the patient. Supplementary active compounds may also be incorporated into the compositions. The “pharmaceutically acceptable carrier” may further include a pharmaceutically acceptable salt of the compound useful within the invention. Other additional ingredients that may be included in the pharmaceutical compositions used in the practice of the invention are known in the art and described, for example in Remington's Pharmaceutical Sciences (Genaro, Ed., Mack Publishing Co., 1985, Easton, PA), which is incorporated herein by reference.

Throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### DESCRIPTION

The present invention utilizes the intrinsic properties of biomaterials to immobilize the bioactive factors present predominantly in PRP and BMAC, in order to prevent their burst release and prolong bioactivity. The immature loss of bioactivity and heterotrophic effects due to burst release of growth factors are well-acknowledged issues with current growth factor-based therapies. Thus, the formulations of the present invention are developed as a thermoresponsive injectable viscous gel or shear-thinning viscoelastic gel that help localize the engineered materials to the injection site. One or more components of the injectable gel may be a component of cartilage ECM that helps re-create the joint homeostasis by encouraging endogenous ECM production.

Accordingly, the present invention is based, in part, on the unexpected results that formulating interleukin-1 receptor antagonist protein (IRAP), alpha-2-macroglobulin, and platelet derived growth factor (PDGF) with an aqueous carrier solution results in an injectable thermoresponsive or shear-thinning gel effective in localized treatment of osteoarthritis. Thus, in various embodiments, the present invention provides a composition comprising at least one recombinant factor (i.e., IRAP and/or PDGF) and a carrier solution comprising at least one selected from hyaluronic acid (HA), chondroitin sulfate (CS), methyl cellulose (MC), and/or two-dimensional silicate nanomaterial. In some embodiments, the two-dimensional silicate nanomaterial is Laponite® (LAP). In some embodiments, the composition comprises an inorganic component. In some embodiments, the composition does not comprise any inorganic components. In some embodiments, the composition is formulated to enable electrostatic binding of the recombinant factors to prevent burst release and/or early loss of bioactivity.

In some embodiments, the composition is an injectable composition. In some embodiments, the composition prevents or reduces HA degradation, prevents or reduces extracellular matrix (ECM) degradation, promotes chondrocyte growth, and/or promotes proliferation to accelerate new ECM synthesis at the injection site. Thus, in some embodiments, the present invention also provides a method of preventing or reducing HA degradation, preventing or reducing ECM degradation, promoting chondrocyte growth, and/or promoting proliferation to accelerate new ECM synthesis at the injection site using the injectable composition of the present invention.

In some embodiments, the composition is an anti-inflammatory composition. Thus, in some embodiments, the present invention also provides a method of treating an inflammatory disease or disorder using the composition of the present invention. In some embodiments, the inflammatory disease or disorder is osteoarthritis. In other embodiments, the present invention also provides a method of treating a degenerative disease or disorder using the composition of the present invention.

### Compositions and Formulations

The present invention provides a thermoresponsive gel composition comprising a carrier solution and at least one recombinant factor. In some embodiments, the composition is formulated to enable electrostatic binding of the recombinant factors to prevent burst release and/or early loss of bioactivity. In some embodiments, the composition is a shear-thinning gel composition. In some embodiments, the thermoresponsive gel maintains a liquid phase at time of injection and subsequently gels at physiologic temperature to remain localized at the treatment site of a subject, such that it can controllably release recombinant factors or other therapeutic agents in vivo.

For example, and without limitation, the recombinant factor may be IRAP, PDGF, alpha-2-macroglobulin or any combination thereof.

The recombinant factor may be formulated at a lower dose or concentration than conventionally required for effectiveness. Similarly, the carrier solution may be formulated at a lower dose or concentration than conventionally required for effectiveness. For example, in some embodiments, one or more of the recombinant factors and the carrier solution may be in the composition at a concentration ranging from about 500 ng/mL to about 200 mg/mL. In some embodiments, one or more of the recombinant factors and the carrier solution may be in the composition at a concentration ranging from about 500 ng/mL to about 1 mg/mL. In some embodiments, one or more of the recombinant factors and the carrier solution may be in the composition at a concentration ranging from about 500 ng/mL to about 1 μg/mL. In some embodiments, the composition comprises between about 1 mg to about 100 mg of IRAP. In some embodiments, the composition comprises between about 10 mg to about 200 mg of PDGF.

Without limitation, the carrier solution may include at least one of HA, CS, MC, chitosan, a two-dimensional silicate nanomaterial or any combination thereof. In various embodiments, the thermoresponsive gel composition comprises a carrier solution comprising any natural or synthetic polymer or combination of polymers which is capable of swelling with water and gelling at a desired temperature and may optionally include a crosslinker. Other non-limiting examples of suitable polymers include, alginate, chitosan, single-arm or multi-arm polyethylene glycol (PEG), collagen, gelatin, starch, agarose, or any combination thereof. The polymer(s) may be present in the thermoresponsive gel at a concentration ranging from about 0.2% to about 20% by weight. For example, the polymer(s) may be present at a concentration of about 0.2%, about 0.5%, about 1%, about 2%, about 3%, about 4%, about 5%, about 10%, about 15%, or about 20%. However, other concentrations are possible, provided the resulting thermoresponsive gel retains the desirable viscosity for injectability at such concentrations of polymer.

In various embodiments, the carrier solution comprises a solvent. In various embodiments, the solvent is a sterile solvent. Examples of such solvents include, but are not limited to, water, sterile water, saline (acidic, basic, or neutral), hydrochloric acid (HCl), dimethyl sulfoxide (DMSO), dimethylformamide (DMF), or any combination thereof. In some embodiments, the composition can further comprise a stabilizer such as Human Serum Albumin (HSA).

In other embodiments, the composition comprises an inorganic component. In some embodiments, the inorganic component is a two-dimensional silicate nanomaterial. In some embodiments, the two-dimensional silicate nanomaterial comprises a negatively charged surface. In some embodiments, the two-dimensional silicate nanomaterial comprises positively charged edges. In some embodiments, the two-dimensional silicate nanomaterial is a two-dimensional nanodisk. In some embodiments, the two-dimensional silicate nanomaterial has an average diameter of about 25 nm and can be in the range of 10 nm to 100 nm. In some embodiments, the two-dimensional silicate nanomaterial has an average thickness of about 2 nm and in the range of 1-2 nm. For example, in some embodiments, the two-dimensional silicate nanomaterial is Laponite® (LAP).

In some embodiments, the composition comprises a carrier solution comprising between about 1% to about 5% w/v of CS. For example, in some embodiments, the composition comprises about 1% w/v CS, about 2% w/v CS, about 3% w/v CS, about 4% w/v CS or about 5% w/v CS.

In some embodiments, the composition comprises a carrier solution comprising between about 1% to about 10% w/v of MC. For example, in some embodiments, the composition comprises about 5% w/v MC, about 10% w/v MC, about 15% w/v MC or about 20% w/v MC.

In some embodiments, the composition comprises a carrier solution comprising between about 0% to about 1.5% w/v of HA. For example, in some embodiments, the composition comprises about 0.2% w/v HA, about 0.5% w/v HA, about 1% w/v HA, about 1.2% w/v HA or about 1.5% w/v HA.

In some embodiments, the composition comprises a carrier solution comprising between about 0.05% to about 2% w/v of inorganic material, such as a two-dimensional silicate nanomaterial (e.g., LAP). For example, in some embodiments, the composition comprises about 0.05% w/v inorganic material, about 0.1% w/v inorganic material, about 0.25-0.5% w/v inorganic material, about 0.5-1% w/v inorganic material or about 2% w/v inorganic material.

Numerous combinations of the recombinant factors and carrier solution are possible and entirely encompassed within the scope of the present disclosure. For example, in some embodiments, the composition does not comprise an inorganic component. Thus, in some embodiments, the composition comprises a recombinant factor comprising IRAP and PDGF and a carrier solution comprising at least one selected from HA, CS, and MC. In some embodiments, the composition comprises a recombinant factor comprising IRAP and a carrier solution comprising at least one selected from HA, CS, and MC. In some embodiments, the composition comprises a recombinant factor comprising PDGF and a carrier solution comprising at least one selected from HA, CS, and MC. In some embodiments, the composition comprises a recombinant factor comprising IRAP and PDGF and a carrier solution comprising CS and MC. For example, in some embodiments, the composition comprises a recombinant factor comprising IRAP and PDGF and a carrier solution comprising 1:1 ratio of CS and MC and can be in the range of 1:1 to 1:10. In some embodiments, the composition comprises a recombinant factor comprising IRAP and a carrier solution comprising CS and MC. In some embodiments, the composition comprises a recombinant factor comprising PDGF and a carrier solution comprising CS and MC.

Further, in some embodiments, the composition comprises a recombinant factor comprising IRAP and PDGF and a carrier solution comprising a two-dimensional silicate nanomaterial (e.g., LAP) and at least one selected from HA, CS, and MC. For example, in some embodiments, the composition comprises a recombinant factor comprising IRAP and PDGF and a carrier solution comprising a two-dimensional silicate nanomaterial (e.g., LAP) and HA. In some embodiments, the composition comprises a recombinant factor comprising IRAP and a carrier solution comprising a two-dimensional silicate nanomaterial (e.g., LAP) and HA. In some embodiments, the composition comprises a recombinant factor comprising PDGF and a carrier solution comprising a two-dimensional silicate nanomaterial (e.g., LAP) and HA.

For example, in some embodiments, the composition is an inorganic component-free composition comprising between about 1 mg to 100 mg of IRAP, between about 10 mg to 200 mg w/v of PDGF, between about 1% to about 5% w/v of CS, and between about 5% to about 20% w/v of MC.

In other embodiments, the composition is an inorganic component-containing composition comprising between about 1 mg to 100 mg w/v of IRAP, between about 5 mg to 100 mg w/v of PDGF, between about 0.2% to about 1.5% w/v of HA, and between about 0.05% to about 2% w/v of LAP.

In some embodiments, the composition is a liquid suitable for injection into a treatment site of a subject between about 0° C. to about 28° C. (e.g., from temperatures cooled on ice, to standard room temperatures). For example, in some embodiments, the composition is a liquid at about 0.5° C., about 1° C., about 2° C., about 3° C., and about 4° C. In some embodiments, the composition is a liquid at about 20° C., about 22° C., about 24° C., about 26° C., and about 28° C. In some embodiments, the composition gels at between about 32° C. to about 45° C. For example, in some embodiments, the composition gels at about 32° C., about 33° C., about 34° C., about 35° C., about 36° C. and about 37° C. Thus, in some embodiments, the composition is a thermoresponsive gel composition capable of transitioning from an injectable, viscous liquid at lower temperatures to an at least partially solidified gel at physiological temperatures post-injection.

In some embodiments, the carrier solution and the recombinant factors can be prepared separately, and then either stored separately or combined. If stored separately before being combined, the carrier solution can be formulated into a suspension, nanosuspension, emulsion, solution, liquid formulation, or gel. If stored separately before being combined, the recombinant factors can be formulated into a powder, granule, suspension, nanosuspension, emulsion, solution, liquid formulation, or gel. If stored separately before being combined, the carrier solution and the recombinant factors may be stored at a temperature low enough to preserve the bioactivity of any active substances (i.e., the recombinant factors), for example at about 4° C. Similarly, the combining of these components may be undertaken at a temperature low enough to prevent the thermoresponsive gel from gelling or solidifying, and low enough to preserve the bioactivity of any active substances (i.e., the recombinant factors). For example, the carrier solution and the recombinant factors may be combined at a temperature of about 4° C. In some examples, the carrier solution and the recombinant factors may be combined at room temperature still without gelling of the thermoresponsive gel. Once combined, the resulting solution may be stored at a low enough temperature to prevent the thermoresponsive hydrogel from gelling or solidifying, such as at 4° C., or, in some embodiments, room temperature. When desired, the combined composition may be injected or otherwise administered to a desired anatomical location in the subject. The physiologic temperature may cause the thermoresponsive gel to undergo a sol-gel reaction and thicken, gellate or otherwise solidify in place. Thus, in some embodiments, the composition is an injectable composition. In some embodiments, the composition is a pharmaceutical composition.

In some embodiments, over time the injected shear-thinning or thermoresponsive gel composition may be absorbed or degraded in vivo. As the shear-thinning or thermoresponsive gel composition is being absorbed or degraded by the body, the composition releases the recombinant factor. Thus, the release rates of the recombinant factor can be adjusted by altering the rate at which the shear-thinning or thermoresponsive gel composition is degraded in vivo, which can be tailored by adjusting the molecular weight of the polymer(s) used to form the carrier solution and the shear-thinning or thermoresponsive gel composition. In general, the larger the molecular weight of the polymer(s) (e.g., MC, CS, and/or HA) used to form the carrier solution and the shear-thinning or thermoresponsive gel composition, the longer it will take for the recombinant factor to be released from the composition. Additionally, the size of the polymer(s) (e.g., MC, CS, and/or HA) used to form the carrier solution may impact how long the shear-thinning or thermoresponsive gel composition takes to degrade in vivo. Thus, for example, two or more types of polymer(s) (e.g., MC, CS, and/or HA) having multiple sizes can be combined so as to produce different release rates of the recombinant factor.

In some embodiments, the composition prevents HA degradation, prevents extracellular matrix (ECM) degradation, modulates chondrocyte growth, and/or modulates proliferation to accelerate new ECM synthesis. In some embodiments, the composition prevents HA degradation, prevents ECM degradation, promotes chondrocyte growth, and/or promotes proliferation to accelerate new ECM synthesis. In some embodiments, the composition is an anti-inflammatory composition.

### Method of Treatment

The present invention also relates, in part, to a method of treating an inflammatory disease or disorder in a subject in need thereof, the method comprising administering to the subject a therapeutic effective amount of at least one composition of the present invention. In various aspects, the present invention also provides a method of alleviating pain associated with an inflammatory disease or disorder in a subject in need thereof, the method comprising administering to the subject a therapeutic effective amount of at least one composition of the present invention. In some embodiments, the inflammatory disease or disorder is an osteoarthritis or degenerative disc disease.

For example, in some embodiments, the present invention provides a method of treating osteoarthritis or alleviating pain associated with osteoarthritis, in a subject in need thereof, the method comprising administering to the subject a therapeutic effective amount of a composition that comprises a recombinant factor comprising IRAP and/or PDGF and a carrier solution comprising HA, CS, MC, and/or two-dimensional silicate nanomaterial.

In other aspects, the present invention also relates, in part, to a method of treating a degenerative disease or disorder in a subject in need thereof, the method comprising administering to the subject a therapeutic effective amount of at least one composition of the present invention.

In various aspects, the present invention also provides a method of alleviating pain associated with a degenerative disease or disorder in a subject in need thereof, the method comprising administering to the subject a therapeutic effective amount of at least one composition of the present invention.

In various aspects, the present invention also relates, in part, to a method of preventing or reducing HA degradation, preventing or reducing ECM degradation, promoting chondrocyte growth, and/or promoting proliferation to accelerate new ECM synthesis in a subject in need thereof, the method comprising administering to the subject a therapeutic effective amount of at least one composition of the present invention.

In some embodiments, the composition is administered to the subject via an injection. In one embodiment, the composition of the present invention is administered via a localized injection of the composition. For example, in one embodiment, the composition of the present invention is administered via an injection of the composition directly into the area of pain. In one embodiment, the composition of the present invention is administered using intramuscular injection. In one embodiment, the composition of the present invention is administered using subcutaneous injection. In one embodiment, the composition of the present invention is administered using intramedullary injection. In one embodiment, the composition of the present invention is administered using intrathecal injection, intra-articular injection or intradiscal injection.

In various embodiments, the composition prevents or reduces HA degradation, prevents or reduces ECM degradation, promotes chondrocyte growth, promotes proliferation to accelerate new ECM synthesis, or any combination thereof at the injection site.

In other embodiments, the composition alleviates pain at the injection site.

In some embodiments, the composition is formulated by combining the carrier solution and the recombinant factor prior to the administration to the subject. For example, in one embodiment, the composition is formulated less than 1 hour prior to the administration to the subject.

In cases of local administration or selective uptake, the effective local concentration of the drug may not be related to plasma concentration. The amount of composition administered will, of course, be dependent on the subject being treated, on the subject's weight, the severity of the affliction, the manner of administration and the judgment of the prescribing physician.

The compositions of the invention may, if desired, be presented in a pack or dispenser device, which may contain one or more unit dosage forms containing the active ingredient. The pack may for example comprise metal or plastic foil, such as a blister pack. The pack or dispenser device may be accompanied by instructions for administration. The pack or dispenser may also be accompanied with a notice associated with the container in form prescribed by a governmental agency regulating the manufacture, use, or sale of pharmaceuticals, which notice is reflective of approval by the agency of the form of the drug for human or veterinary administration. Such notice, for example, may be the labeling approved by the U.S. Food and Drug Administration for prescription drugs, or the approved product insert. Compositions comprising a compound disclosed herein formulated in a compatible pharmaceutical carrier may also be prepared, placed in an appropriate container, and labeled for treatment of an indicated condition.

### Kits

It is further envisioned that the compositions and methods described herein can be embodied in the form of a kit or kits. A non-limiting example of such a kit is a kit for treating an inflammatory disease or disorder, such as osteoarthritis, or degenerative disease or disorder or a kit for alleviating pain associated with an inflammatory disease or disorder, such as osteoarthritis, or degenerative disease or disorder, the kit may optionally include a carrier solution and optionally a recombinant factor in separate containers, where the containers may or may not be present in a combined configuration. Many other kit components are contemplated, such as a kit further including ice, dry ice, a thermos, or other means for keeping the composition below room temperature. The kits may further include instructions for using the components of the kit to practice the subject methods. The instructions for practicing the subject methods are generally recorded on a suitable recording medium. For example, the instructions may be present in the kits as a package insert or in the labeling of the container of the kit or components thereof. In other embodiments, the instructions are present as an electronic storage data file present on a suitable computer readable storage medium, such as a flash drive. In other embodiments, the actual instructions are not present in the kit, but means for obtaining the instructions from a remote source, such as via the internet, are provided. An example of this embodiment is a kit that includes an internet address where the instructions can be viewed and/or from which the instructions can be downloaded. As with the instructions, this means for obtaining the instructions is recorded on a suitable substrate.

It will be understood by those of skill in the art that numerous and various modifications can be made without departing from the spirit of the present disclosure. Therefore, it should be clearly understood that the forms disclosed herein are illustrative only and are not intended to limit the scope of the present disclosure.

### EXPERIMENTAL EXAMPLES

The invention is further described in detail by reference to the following experimental examples. These examples are provided for purposes of illustration only and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the compositions of the present invention and practice the claimed methods. The following working examples, therefore, specifically point out the preferred embodiments of the present invention and are not to be construed as limiting in any way the remainder of the disclosure.

### Therapeutic Injection for Osteoarthritis

Interleukin-1 receptor antagonist protein (IRAP) and platelet derived growth factor (PDGF) have been identified as the key anti-inflammatory factors in PRP and BMAC therapies and crucial for efficacy of clinical treatment. For this reason, the present invention demonstrates the unexpected benefit and improvement of an injectable gel loaded with recombinant factors for the treatment of osteoarthritis.

Both IRAP and PDGF are key components for PRP and BMAC. IRAP provides the anti-inflammatory environment to prevent or reduce HA and extracellular matrix (ECM) degradation. PDGF can promote the chondrocyte growth and proliferation to accelerate new ECM synthesis at the injection site. It should be appreciated that any of the compositions or products described herein can come with a similar amount of growth factor, but the volume of the final product can vary depending on clinical needs. HA provides the visco-supplementation to the arthritic joint and is currently available as therapeutics for osteoarthritic and other degenerative diseases. CS is a sulfated glycosaminoglycan that forms an important component of cartilage ECM.

In a first example, an injectable gel is formed with the inclusion of LAP. LAP is a two-dimensional (2D) silicate nanomaterial with a thickness of 2 nm and diameter of 25 nm. The surface and edges of these 2D nanodisks are negatively and positively charged, respectively. Due to this unique heterogenicity in charge distribution, these nanomaterials quickly form an aqueous gel that can be injected using smaller gauge needles and catheters. These charges are utilized to electrostatically bind the growth factors. The gel forming property further help localize the formulation to the injection site. The carrier comes with intrinsic osteo-chondrogenic potential.

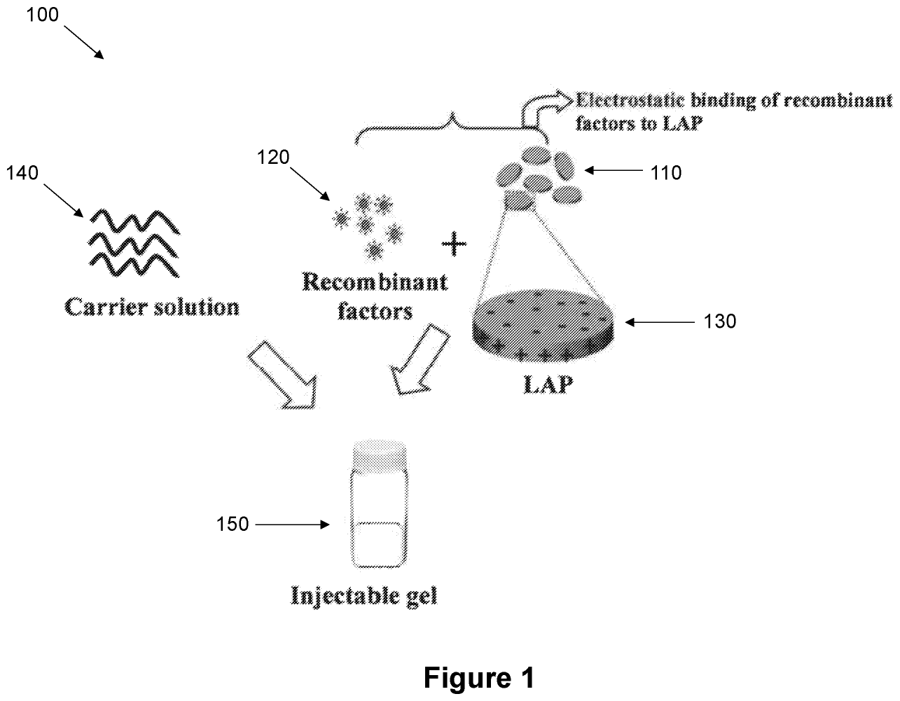

As shown in FIG. 1, a system 100 forming injectable gel 150 as described herein includes a first carrier solution option that is specifically engineered to enable electrostatic binding of the recombinant factors to prevent the burst release and early loss of bioactivity. Specifically, the carrier solution includes Laponite® (LAP) 110, which is combined with recombinant factors IRAP and/or PDGF 120 to obtain the desired electrostatic binding of recombinant factors 130. This first carrier solution option further includes any combination of HA, chondroitin sulfate (CS), and/or methyl cellulose (MC) 140 to form the injectable gel 150. The final product is a viscoelastic gel 50 that remains suitably fluid at the time of injection to the osteoarthritic site of a subject, while subsequently gelating at the treatment site to effectuate a slow, localized release of the therapeutic agents.

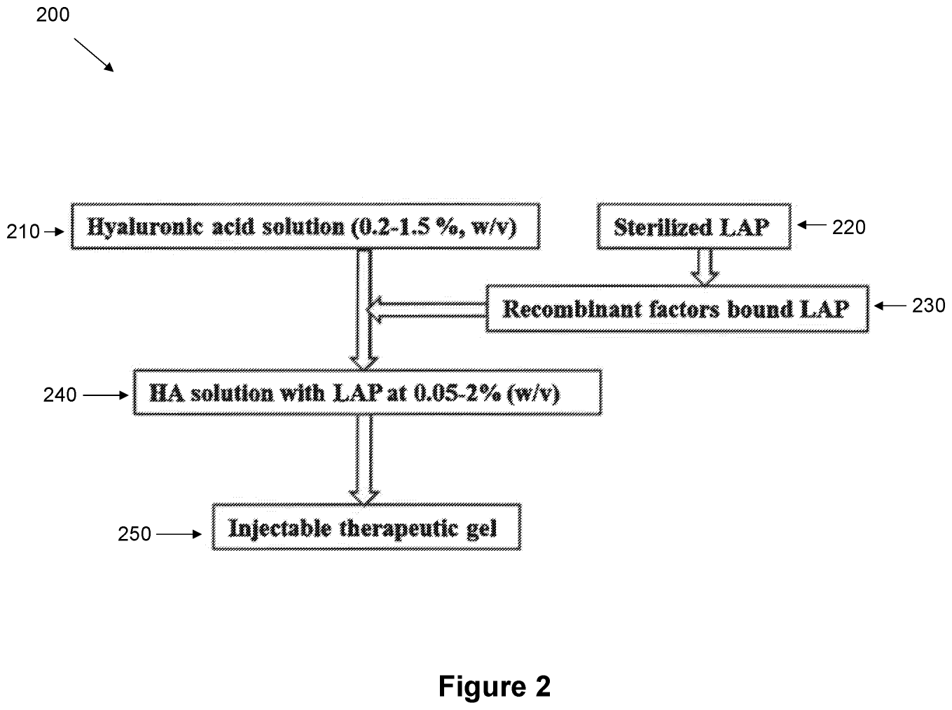

FIG. 2 is a flowchart showing method and components 200 in the formation of the injectable therapeutic gel of FIG. 1. First, at 210, sterile HA solution is prepared by dissolving irradiation-sterilized HA in sterile water under aseptic conditions (0.2%-1.5% w/v). At 220, LAP powder is irradiation-sterilized prior to use. At 230, the sterilized LAP powder is combined with one or more recombinant factors. At 240, sterilized LAP powder and recombinant factor is added to the sterile HA solution at the concentration ranging from 0.05% to 2% (w/v). LAP is highly hydrophilic. Therefore, uniformly dispersed HA-LAP gels are developed by applying mechanical agitation. This viscoelastic gel has a shear-thinning property that enable an easy injection but forms re-conforms to gel structure once injected.

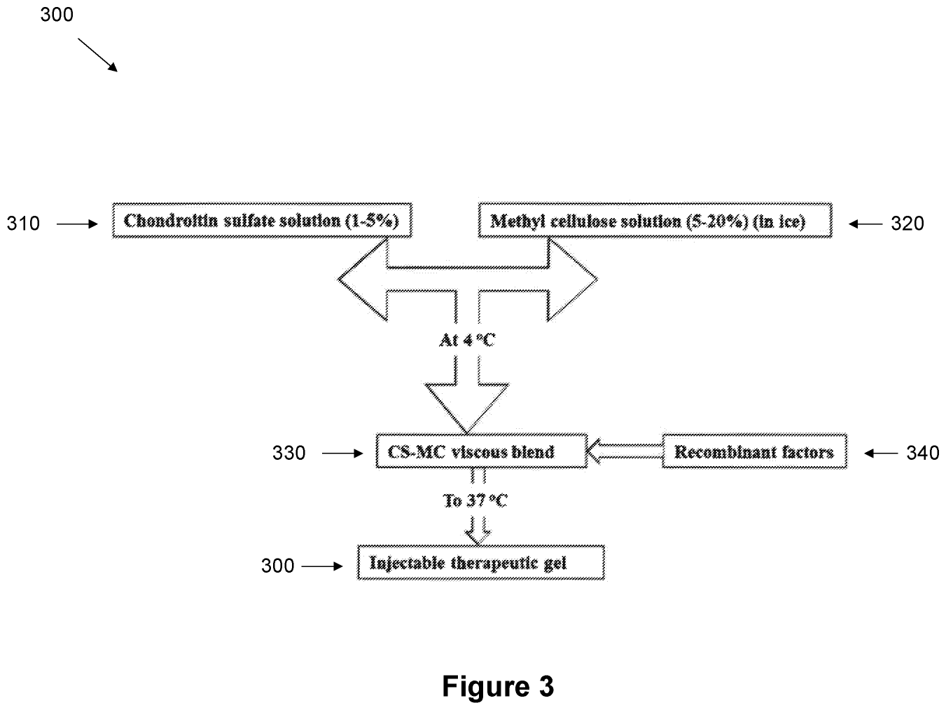

FIG. 3 is a flowchart showing a second method and components 300 in the formation of an injectable therapeutic gel utilizing CS and MC as carriers. CS is a key ECM component of cartilage and plays an important role in maintaining high hydration level due to the presence negatively charged sulfate groups. Both CS and MC powders are sterilized through irradiation before preparing the solution. The solutions are prepared individually at the concentration of 1-5% for CS (310) and 5-20% for MC (320). MC solution is prepared under ice and maintained at the same temperature. At 330, CS and MC solutions are mixed at 1:1 ratio and maintained at 4° C. before injection. At 340, recombinant factors are added to the CS-MC blend. At 350, the resultant injectable therapeutic gel stays as a viscous liquid at 4° C., enabling an easy injection and subsequently undergoes gelation once the transition of temperature occurs at physiological temperature. All the solutions are prepared in sterile water and under aseptic conditions.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

1. A composition comprising:
a recombinant factor, wherein the recombinant factor comprises at least one selected from the group consisting of interleukin-1 receptor antagonist protein (IRAP) and platelet derived growth factor (PDGF); and P1 a carrier solution, wherein the carrier solution comprises a solvent and at least one selected from the group consisting of hyaluronic acid (HA), chondroitin sulfate (CS), methyl cellulose (MC), and two-dimensional silicate nanomaterial;
wherein the composition is a thermoresponsive gel or shear-thinning gel.

2. The composition of claim 1, wherein the recombinant factor comprises IRAP and PDGF.

3. The composition of claim 1, wherein the composition comprises an inorganic component.

4. The composition of claim 3, wherein the composition comprises a carrier solution comprising a two-dimensional silicate nanomaterial and at least one selected from the group consisting of HA, CS, and MC.

5. The composition of claim 1, wherein the two-dimensional silicate nanomaterial is Laponite® (LAP).

6. The composition of claim 5, wherein the composition comprises a carrier solution comprising between about 0.05% to about 2% w/v of LAP and between about 0.2% to about 1.5% w/v of HA.

7. The composition of claim 1, wherein the composition does not include an inorganic component.

8. The composition of claim 7, wherein the composition comprises a carrier solution comprising at least one selected from the group consisting of HA, CS, and MC.

9. The composition of claim 8, wherein the composition comprises a carrier solution comprising between about 1% to about 5% w/v of CS and between about 5% to about 20% w/v of MS.

10. The composition of claim 1, wherein the composition gels at physiological temperatures.

11. The composition of claim 1, wherein the composition is a liquid at between 0° C. and room temperature.

12. The composition of claim 1, wherein the composition is an injectable liquid composition.

13. The composition of claim 1, wherein the composition is an anti-inflammatory composition.

14. The composition of claim 1, wherein the composition prevents or reduces HA degradation, prevents or reduces ECM degradation, promotes chondrocyte growth, promotes proliferation to accelerate new ECM synthesis, or any combination thereof.

15. A method of preventing or reducing HA degradation, preventing or reducing ECM degradation, promoting chondrocyte growth, promoting proliferation to accelerate new ECM synthesis, or any combination thereof in a subject in need thereof, wherein the method comprises administering to the subject a therapeutic effective amount of at least one composition of claim 1.

16. A method of treating osteoarthritis in a subject in need thereof, wherein the method comprises administering to the subject a therapeutic effective amount of at least one composition of claim 1.

17. A method of treating osteoarthritis in a subject in need thereof, wherein the method comprises administering to the subject a therapeutic effective amount of a composition comprising:
a recombinant factor, wherein the recombinant factor comprises at least one selected from the group consisting of IRAP and PDGF; and
a carrier solution, wherein the carrier solution comprises a solvent and at least one selected from the group consisting of HA, CS, MC, and two-dimensional silicate nanomaterial;
wherein the composition is a thermoresponsive gel or shear-thinning gel.

18. The method of claim 17, wherein the composition is administered to the subject via an injection.

19. The method of claim 18, wherein the composition prevents or reduces HA degradation, prevents or reduces ECM degradation, promotes chondrocyte growth, promotes proliferation to accelerate new ECM synthesis, or any combination thereof at the injection site.

20. The method of claim 17, wherein the composition is formulated less than 1 hour prior to the administration to the subject.
